(19) United States
(12) Patent Application Publication
(10) Pub. No.: US 2020/0376100 A1
(43) Pub. Date: Dec. 3, 2020

(54) IMMUNOTHERAPY OF SOLID TUMORS BY VACCINATION WITH TRANSGENIC LEISHMANIA EXPRESSING CANCER VACCINES AND WITH RECOMBINANT VACCINES WITH LEISHMANIA AS ADJUVANTS

(71) Applicant: Rosalind Franklin Univ. of Medicine and Science, North Chicago, IL (US)

(72) Inventors: Kwang-Poo Chang, Kenilworth, IL (US); Bala K. Kolli, North Chicago, IL (US)

(21) Appl. No.: 16/888,576

(22) Filed: May 29, 2020

Related U.S. Application Data
(60) Provisional application No. 62/855,621, filed on May 31, 2019.

(51) Int. Cl. A61K 39/008; A61K 39/395

(52) U.S. Cl. CPC A61K 39/008; A61K 2039/5154; A61K 39/39558

(57) ABSTRACT

Disclosed herein are methods of vaccination against cancer by providing a sample of a photo-inactivated Leishmania optionally transgenically modified to express a cancer antigen such as alpha-enolase; optionally providing an effective amount of an alpha-enolase; and delivering an effective amount of the sample and/or the alpha-enolase to a patient in need thereof.

### CROSS REFERENCE TO RELATED APPLICATIONS

The present invention claims priority to and the benefit of U.S. Provisional Patent Application No. 62/855,621 filed May 31, 2019, the contents of which are incorporated in their entirety herein by reference and made a part hereof.

### FIELD OF THE INVENTION

The present invention provides an immunotherapy of a cancerous tumor and more specifically immunotherapy of cancer by photodynamic vaccination using transgenically modified Leishmania.

### BACKGROUND OF THE INVENTION

By their innate ability to dwell in the endosome/phagolysosomes of antigen-presenting cells, Leishmania are a suitable carrier for vaccine delivery to elicit robust immunity. The efficacy of Leishmania to serve in that capacity is preserved when rendered non-viable and thus safe to use by photodynamic therapy or treatment (PDT). The unique feature of PDT makes this possible. Exposure of photosensitized Leishmania to light in presence of oxygen produces a burst of very short-lived, albeit highly reactive radicals, which kills them instantly and completely, leaving their antigenicity and adjuvanticity unscathed to serve as carrier to deliver transgenic vaccines and adjuvants.

A novel cell-mediated immunotherapy is being developed according to the Leishmania strategy of vaccine delivery (Chang et al., 2016 Parasit Vectors. 9:396) against difficult-to-cure diseases, e.g., canine leishmaniasis. The current clinical management of this disease entails prolonged treatments of sick dogs for 30 days with heavy daily dosage of very toxic drugs (antimonials/miltefosine) followed by a daily maintenance dose of allopurinol for life. Still, relapses of the disease are frequent (up to 50%) within the first year (Manna et al., 2015 Parasit Vectors. 8: 289).

Commonly assigned U.S. Pat. Nos. 7,261,887; 7,238,347, 9,327,017, U.S. Patent Publication No. 2017/0042989 disclose using leishmania as a carrier for vaccine delivery or for the delivery of peptides and proteins, and U.S. Patent Publication No. 2018/0318408 is directed to immunotherapy of canine leishmaniasis. All of these documents are incorporated herein in their entirety by reference and made a part hereof.

### SUMMARY OF THE INVENTION

Disclosed herein is a method for vaccination against cancer. The method includes the steps of: providing a sample of a photo-inactivated Leishmania; providing a sample of GST-alpha-enolase; mixing the photo-inactivated Leishmania with the GST-alpha-enolase sample to form a vaccine mixture; and, delivering an effective amount of the vaccine mixture to a patient in need thereof.

Also disclosed herein is a container of a solution for vaccination against cancer. The vaccination solution is a mixture of a photo-inactivated Leishmania and a GST-alpha-enolase.

A method for treating a solid tumor in a patient includes: obtaining a sample of peripheral blood mononuclear cells (PBMC) cells from the patient; isolating CD14 positive monocytes from the PBMC sample; maturing the CD14 positive monocytes into mature dendritic cells; co-cultivating the mature dendric cells with isolated T cells to activate CD8+ T cells and cytotoxic T lymphocytes activity; providing a sample of a photo-inactivated Leishmania; mixing the mature dendritic cells with the Leishmania sample to form a vaccination solution; and, delivering an effective amount of the vaccination solution to the patient.

### BRIEF DESCRIPTION OF THE DRAWINGS

FIG. 1 shows three schemes of Leishmania-based photodynamic vaccination in vitro.

FIG. 2 shows a diagrammatic depiction of presentation of Leishmania-delivered vaccines by antigen presenting cells.

FIG. 3 is a diagrammatic view of dendritic cell (DC)-based immunotherapy protocols.

FIG. 4 is a diagrammatic representation of an experimental scheme.

FIG. 5 shows a graph of tumor size vs. time in days after tumor inoculation to show the effects on tumor suppression using a Leishmania adjuvant experimental sample vs. a control sample.

FIG. 6 has a first graph on the left of body weight vs. time in days after inoculation in the control sample (LS+GST) and a second graph on the right of body weight vs. time in days after inoculation in the experimental sample (LS+GST-mENO1).

FIG. 7 shows the significance in difference in tumor sizes in the mice treated with the control sample (LS+GST) (FIG. 7A); in mice treated with the experimental sample (LS+GST-mENO1) (FIG. 7B), and a comparison of resected tumors in the control group on top and the experimental group below (FIG. 7C).

FIG. 8 shows diagrammatically an experimental protocol for immunization of mice and transfer of immunity with splenic cells.

FIG. 9 shows a graph of tumor size vs. time in days after inoculation and shows a significant suppression in tumor by the experimental solution.

FIG. 10 shows two graphs of weight vs. time in days after inoculation showing there was no significant impact on the weight of the mice in the study.

FIG. 11 shows resected tumors from mice in the control group (top) and the experimental group (bottom) showing a significant smaller size in the experimental group over the control group.

### DETAILED DESCRIPTION OF THE INVENTION

While this invention is susceptible of embodiments in many different forms, and will be described herein in detail, specific embodiments thereof with the understanding that the present disclosure is to be considered as an exemplification of the principles of the invention and is not intended to limit the invention to the specific embodiments illustrated.

Antigen-specific vaccination remains to be an option for tumor immunotherapy. The delivery of vaccines for this approach includes the strategies of using attenuated bacterial and viral constructs, e.g. Listeria and Vaccinia. Leishmania are parasitic protozoa, which are uniquely favorable for such use as a universal platform to deliver vaccines for disease prevention and therapy. Of particular relevance is the human cutaneous Leishmania, which causes innocuous, self-resolving skin infection. Life-long immunity develops after its spontaneous cure, indicative of not only the presence of vaccines in Leishmania against leishmaniasis but also adjuvanticity critical for effective vaccination against this and other diseases, e.g. malignancy. Leishmania is a vaccine carrier of high efficacy with eukaryotic translational machineries and post-translational mechanisms for correctly expressing multiple foreign vaccines in abundance. Additionally, Leishmania are inherently endowed with multifarious molecules to protect endogenous vaccines and target them specifically to antigen presenting cells (APC), i.e., dendritic cells (DC) and macrophages—the exclusive host cells for the residence of these parasites in natural infection.

Leishmania surface lipoglycoconjugates are attributable to these activities and are also structurally similar to some adjuvants known to enhance the vaccine effectiveness. Leishmania are intrinsically safe. They produce no toxins and show no human toxicity, as indicated by their extensive use after chemical or physical inactivation in Leishmanin skin test for delayed type hypersensitivity and in several large-scale vaccine trial attempts. We have developed novel strategies to completely inactivate Leishmania for safety assurance with the preservation of its adjuvanticity as a vaccine carrier. Dual suicidal mechanisms were installed to accomplish this by genetic and chemical engineering of Leishmania for light-inducible ¹O₂ initiated oxidative inactivation. The safety and efficacy of such inactivated Leishmania have been demonstrated by immunization of animals, producing no adverse effects, but prophylactic activities experimentally against both cutaneous and visceral leishmaniasis, and immunotherapeutic activities clinically against drug-incurable canine leishmaniasis. Moreover, Leishmania transgenically made to express ovalbumin (OVA) was shown to deliver this antigen, more effectively after photodynamic inactivation, to DC for processing and presentation to activate OVA epitope-specific T cells in vitro.

Human cancer vaccine candidates have been successfully expressed in transgenic Leishmania, including alpha-enolase (ENO1). These inactivated Leishmania produced dramatic tumor-suppressing activities by immunotherapy in mouse models mimicking human lung and pancreatic cancer. In active immunotherapy, frozen samples of photo-inactivated Leishmania were used as adjuvants for immunizations with recombinant mouse ENO1 (mENO1) against murine tumors. Similar samples of the human ENO1 (hENO1)-expressing Leishmania were used alone for immunizations of BALB/c mice followed by adaptive transfer of immunity with their splenic cells into immunocompromised mice bearing human tumor. Such photodynamic vaccination was also carried out similarly in KKPC transgenic mice against spontaneously and rapidly developed pancreatic tumor. Tumor antigens have been delivered to patients' DC in vitro by different ways, for example, via conjugation with cell-penetrating peptides and adeno-associated virus vectors for ex vivo vaccination to activate CD8+ T cells for CTL activities of anti-tumor immunity. Such protocols are being adapted for use with inactivated hENO1-Leishmania toward DC-based immunotherapy of human lung cancer.

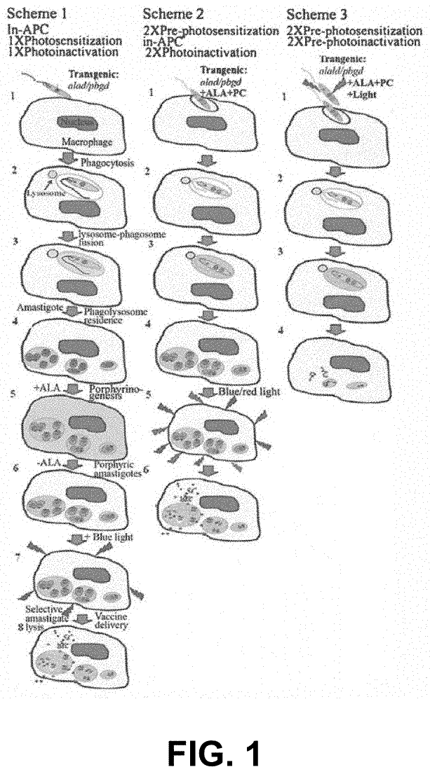

FIG. 1 shows three schemes for Leishmania-based photodynamic vaccination in vitro. The Leishmania-delivered vaccines include the transgenic alad/pbgd porphrinogenic Leishmania transgenically modified to express an antigen of a solid tumor e.g., alpha-enolase. Such tumors include pancreatic cancer tumors and lung cancer tumors for example. The transgenic:alad/pbgd, Porphyrinogenic Leishmania are transfected with two mammalian cDNAs encoding the 2nd and 3rd enzymes in the heme biosynthetic pathway, rendering them susceptible to delta-aminolevulinate (ALA)-induced neogenesis of uroporphyrin (URO). The PC used is Si-phthalocyanine photosensitizer. The step of light illumination is shown with blue and red lightening symbols, blue (400-500 nm wavelength) and red (˜600 nm wavelength) for excitation of URO and PC, respectively.

FIG. 1 shows Scheme 1 includes a single photosensitization and a single photoinactivation of an antigen presenting cell (APC). Steps 1-2 show phagocytosis of porphyrinogenic, but untreated Leishmania by APC. Step 3 shows fusion of a Leishmania-containing phagosome with a lysosome. Step 4 shows Leishmania differentiation into amastigotes and their replication in the phagolysosomes. Step 5 shows exposure of the parasitized APC to ALA, resulting in porphyrinogenesis of both APC and phagolysosomal amastigotes. Step 6 shows removal of ALA, resulting in disappearance of porphyrins from the APC and persistence of URO in the amastigotes. Steps 7-8 show illumination of these APC resulting in selective lysis of URO-loaded amastigotes, releasing vaccines into the phagolysosomes and the cytosol.

FIG. 1 also shows Scheme 2, which is the same as Scheme 1, except that the porphyrinogenic Leishmania are doubly PS-sensitized with ALA and PC in the dark before use for infecting APC. Steps 1-4, as described for Scheme 1, except that the Leishmania are pre-loaded with URO and PC, hence no further ALA treatment is required. Steps 5-6 show illumination of the infected cells with blue and red light to excite URO and PC, lysing amastigotes with singlet oxygen and other ROS generated for releasing vaccines in APC.

FIG. 1 also shows Scheme 3 which is the same as Schemes 1-2, except that the Leishmania are pre-PS-sensitized and pre-photo-inactivated before use for vaccine delivery to APC. Steps 1-4 show the uptake of oxidatively photo-inactivated Leishmania by APC, lysosome-phagosome fusion and their lysis to release vaccines as described

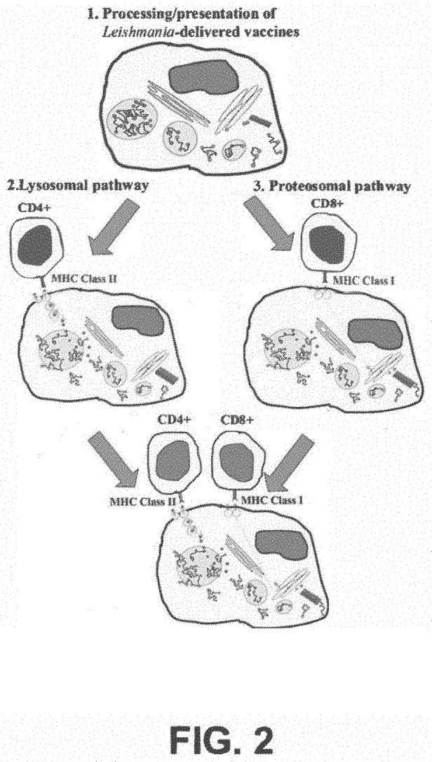

FIG. 2, part 1, shows the processing and presentation of a Leishmania-delivered vaccines of the present invention by antigen-presenting cells. FIG. 2, part 2, shows antigen presentation by lysosomal pathway via MHC Class II for activation of CD4+ T cells. FIG. 2, part 3, shows antigen presentation by proteosomal pathway via MHC Class I for activation of CD8+ T cells.

A sample of transgenic alad/pbgd porphrinogenic Leishmania transgenically modified to express an antigen of a solid tumor e.g., alpha-enolase can be suspended in a suitable solution. The solution can be liquid, frozen or lyophilized and placed in a container for later use, sale, and transport for experimental and commercial purposes. Suitable containers can be rigid or flexible. Rigid containers include glass and plastic jars, vials, ampules for example. Suitable plastics include polyolefins, polystyrenes, polyamides, polyesters, polyvinyl chloride, cyclic olefin copolymers and others known to a person of ordinary skill in the relevant art. Preferably, the rigid containers will maintain their shape upon draining.

Flexible containers include those containers made of polymeric materials, such as polyvinyl chloride, polyolefins, polyamides, polyesters, polystyrene, and the like. Suitable containers include those commonly used to contain fluids for intravenous delivery to a patient. The flexible container can be filled and emptied through a closure such as a spout or port tube. Preferably, the flexible container will collapse upon draining.

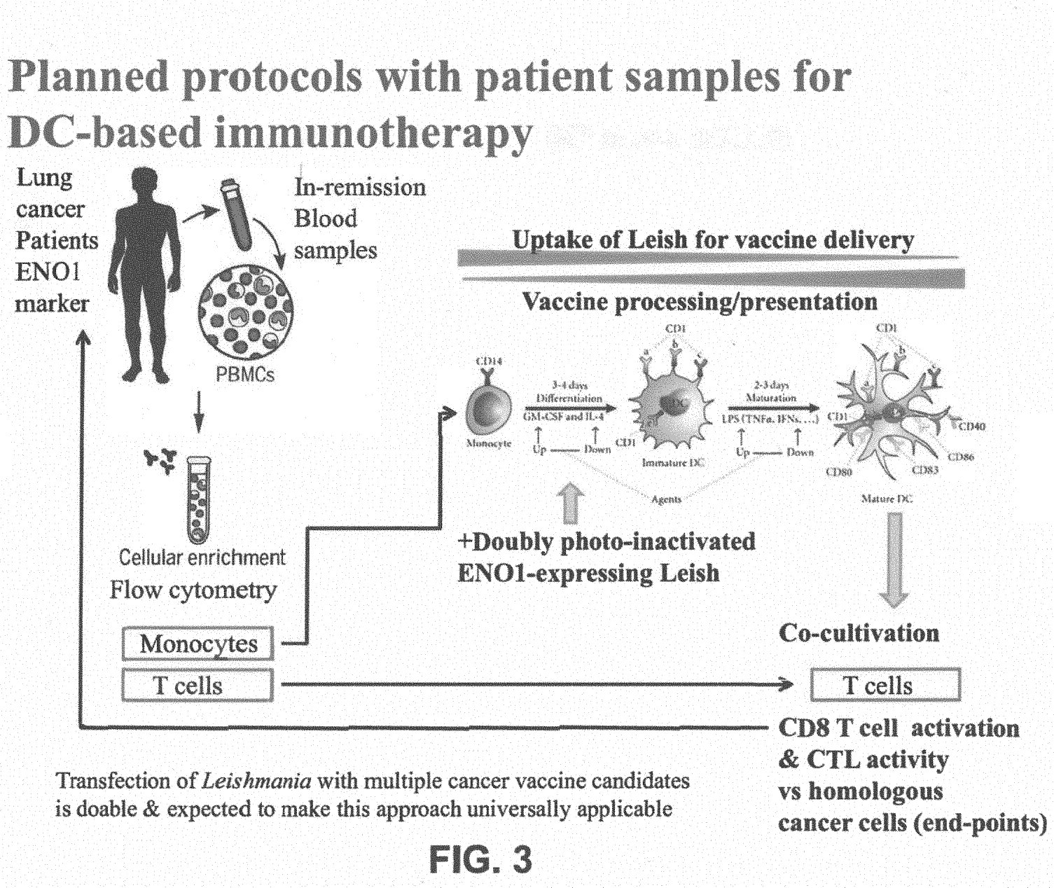

FIG. 3 shows a diagram of planned protocols with patient samples for DC-based immunotherapy. Blood samples will be collected from lung cancer patients with an ENO1 marker and peripheral blood mononuclear cells (PBMC) isolated. The PBMC sample will be subjected to cellular enrichment and subjected to flow cytometry to isolate CD14 monocytes and CD8+ T cells. The monocytes are contacted with an effective amount of the doubly photo-inactivated ENO1 Leishmania described herein.

The CD14 monocytes generate immature DCs upon exposure to granulocyte-macrophage colony stimulating factor (GM-CSF) and interleukin 4 (IL4) after a period of 3-4 days. The immature DCs are induced to maturity by exposure to lipopolysaccharide LPS (TNFα, IFNs, etc.) for 2-3 days. The mature DCs are co-cultivated with the isolated T cells to activate CD8+ T cells and cytotoxic T lymphocytes activity. Homologous cancer cells (end-points) are used as a control. The activated T cells are reinfused to the patient for immunotherapy of the lung cancer.

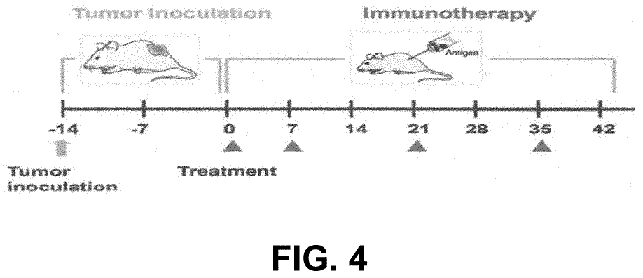

Experiment 1 is to determine the effective adjuvanticity of photodynamically inactivated Leishmania with recombinant enolase-1 peptide as a vaccine for immunotherapy of a tumor produced by murine lung cancer cells in a mouse model. The results are shown in FIGS. 4-7.

FIG. 4 shows an experimental scheme for tumor establishment and immunotherapeutic treatment in ten BALB/c mice (25 gm). Each mouse received i.p. syngenic ML-1 cancer cells (Caliper Life Sciences, Alameda, Calif.) grown in DMED+5% FBS at 10⁵ cells/mouse for 2 wks (orange arrow). They were then divided into 2 groups with 5 mice per group. Five mice in the experimental group were then each treated with 4× immunotherapy at the times as indicated (day 0, 7, 21 and 35) with a mixture of vaccine, i.e. GST-recombinant mouse alpha-enolase (GST-mENO1, 25 ug) and adjuvant, i.e. frozen photodynamically inactivated Leishmania (LS, 10⁷ cells) (loaded with 1 uM amino-phthalocyanine followed by exposure the red light). The Control group was simultaneously and similarly set up, except using GST alone instead of GST-mENO1. GST means glutathione S transferase.

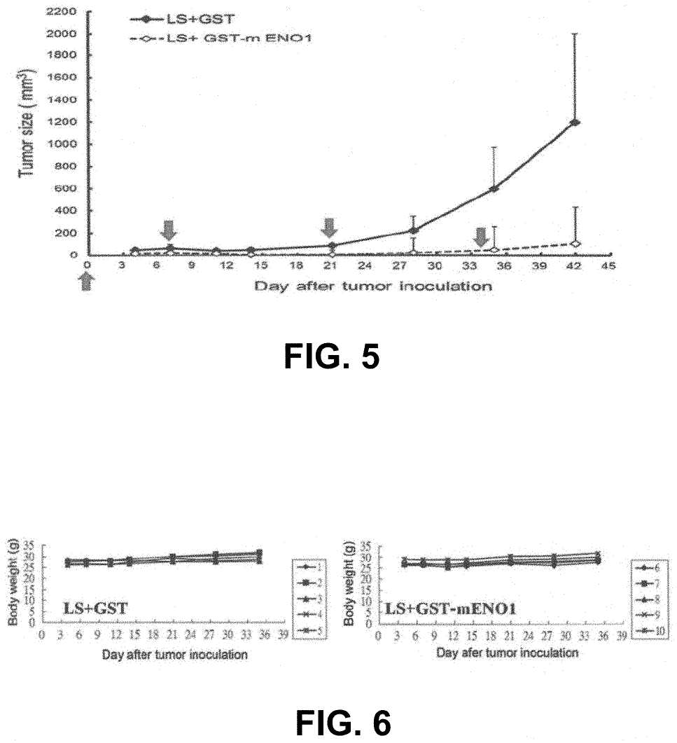

FIG. 5 is a graph of tumor size vs. time in days after inoculation in the control group (LS+GST) and the experimental group (LS+GST-mENO1). Tumor size was measured at the time points as indicated by the arrows. The value at each point represents a mean of the five mice in the group, with standard deviation shown. The results show significant tumor suppression in the experimental group over the control group.

FIG. 6 shows two graphs of body weight vs. time in days after inoculation. In both the control group (graph on the left) and the experimental group (graph on the right) shows there is no significant difference in the body weight of the mice in each group.

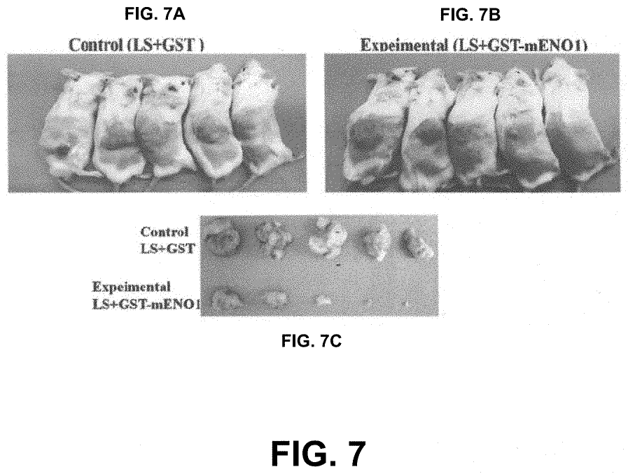

FIG. 7 shows the experimental group tumor sizes were significantly smaller than those of the control group (FIG. 7C). FIGS. 7A and 7B shown the mice after tumor resection in the control group and the experimental group respectively.

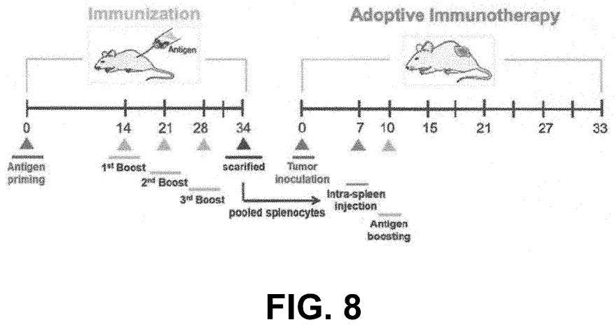

Experiment 2 is to determine the efficacy of the delivery of human lung cancer vaccine by photodynamically inactivated Leishmania transfectants for adoptive immunotherapy in a mouse model. The results are shown in FIGS. 8-11.

FIG. 8 shows the experimental protocol for immunization and adoptive immunotherapy of five BALB/c mice, each immunized with frozen sample of photodynamically inactivated Leishmania transfectants expressing human enolase1 (hENO1) at 10⁷ cells per mouse and boosted 3× on days 14, 21 and 28 (orange triangle, left panel). The control group consisted of another 5 BALB/c mice, which were processed in exactly the same way, except for immunization with photodynamically inactivated mock transfectants with empty plasmids. On day 34, all mice were sacrificed and their splenic cells from respective groups pooled for adoptive transfer of immunity intrasplenically into an equivalent number of syngenic NOD/SCID mice (right panel, Adoptive immunotherapy). These recipient mice were inoculated i.p. 1 wk earlier with human lung adenocarcinoma CL1-5F4 cell line grown in vitro in RPMI 1640+10% FBS and boosted once 3 days after adoptive transfer of splenic cells. All mice were subjected to inspection for development of tumors.

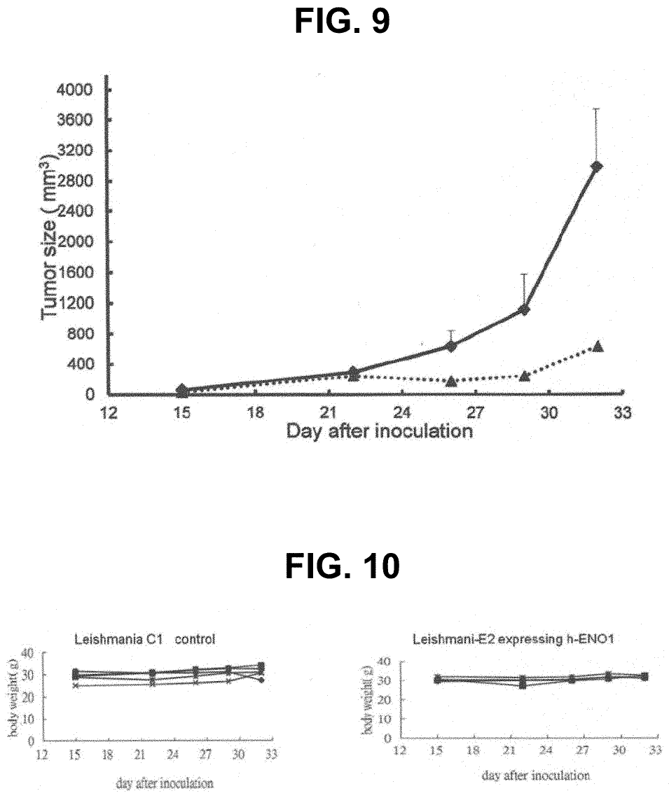

FIG. 9 is a graph of tumor size vs. time in days after inoculation and the control group results are shown in a solid line and the results for the experimental group are shown by the dotted line. The results show a significant suppression in the sizes of the tumors in the experimental group over the control group.

FIG. 10 shows graphs for the control group (left) and the experimental group (right) of body weight vs. time in days after inoculation. There was no significant difference in the body weights of the animals in the control group and the experimental group.

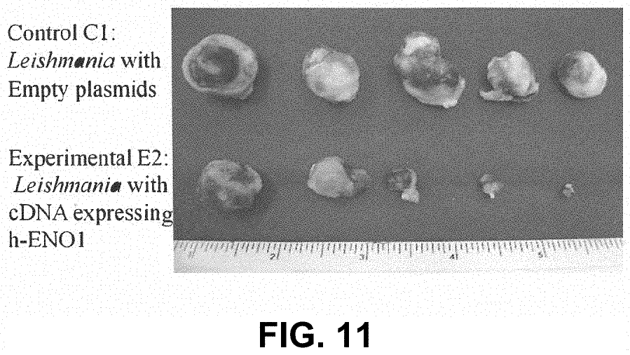

FIG. 11 shows resected tumors from the control group (top) and the experimental group (bottom). The tumors of the experimental group are significantly smaller than the tumors of the control group.

Thus, there are several methods of using the photo-inactivated, porphyric, ENO1 expressing Leishmania in treating mammalian patients. A first method is for vaccination against cancer including the steps of: providing a sample of a photo-inactivated Leishmania; providing an effective amount of an alpha-enolase; and delivering an effective amount of the sample and the alpha-enolase to a patient in need thereof.

Another method for vaccination against cancer includes the steps of: providing a sample of a photo-inactivated Leishmania transgenically modified to express alpha-enolase; and delivering an effective amount of the sample to a patient in need thereof. In one form of the invention the Leishmania are transgenically modified to be susceptible to delta-aminolevulinate for porphyrinogenesis.

Another method for vaccination against cancer includes the steps of: providing a sample of a photo-inactivated Leishmania transgenically modified to express alpha-enolase; delivering an effective amount of the sample to an animal to develop an immunity to alpha-enolase; obtaining a sample of cells from the animal; and, delivering an effective amount of the cells from the animal to a patient in need thereof to transfer the immunity to the patient.

In any of the methods, it is preferred the Leishmania have been transgenically modified with a dual suicidal mechanism.

Another method is for treating a solid tumor in a patient including: obtaining a sample of antigen presenting cells from the human patient; in vitro exposure of the cell sample to an effective amount of Leishmania transgenically modified to express an antigen of the solid tumor to form a vaccinated sample; and, delivering an effective amount of the vaccinated sample to the patient for clearing of the tumor.

The Leishmania-based vaccine to solid tumors can be provided in a container having walls defining a chamber; and a solution in the chamber having a suspension of an effective amount of a Leishmania-based vaccine having Leishmania transgenically modified with a dual suicidal mechanism and to express an antigen of the solid tumor, such as alpha-enolase. The container can have flexible walls or rigid walls.

The appended claims should be construed broadly and in a manner consistent with the spirit and the scope of the invention herein.

We claim:

1. A method for vaccination against cancer comprising:
providing a sample of a photo-inactivated Leishmania;
providing a sample of GST-alpha-enolase;
mixing the photo-inactivated Leishmania with the GST-alpha-enolase sample to form a vaccine mixture; and,
delivering an effective amount of the vaccine mixture to a patient in need thereof.

2. The method of vaccination of claim 1 further comprising the step of loading the photo-inactivated Leishmania with a photosensitizer.

3. The method of vaccination of claim 2 further comprising exposing the photo-inactivated Leishmania with a photosensitizer to red light.

4. The method of vaccination of claim 1 wherein the photo-inactivated Leishmania are transgenically modified to render them susceptible to porphyrinogenesis.

5. The method of vaccination of claim 1 wherein the photo-inactivated Leishmania are transgenically modified to express alpha-enolase.

6. The method of vaccination of claim 1 wherein the photo-inactivated Leishmania are transgenically modified with a dual suicidal mechanism.

7. The method of vaccination of claim 1 wherein the cancer is a solid tumor expressing alpha-enolase.

8. A container of a solution for vaccination against cancer comprising:
a mixture of a photo-inactivated Leishmania and a GST-alpha-enolase.

9. The container of claim 8 wherein the container has flexible side walls.

10. The container of claim 9 wherein the flexible side walls are made from a plastic.

11. The container of claim 8 wherein the container has rigid side walls.

12. The container of claim 8 wherein the photo-inactivated Leishmania are transgenically modified to render them susceptible to porphyrinogenesis.

13. The container of claim 8 wherein the photo-inactivated Leishmania are transgenically modified to express alpha-enolase.

14. The container of claim 8 wherein the photo-inactivated Leishmania are transgenically modified with a dual suicidal mechanism.

15. A method for treating a solid tumor in a patient comprising:
obtaining a sample of peripheral blood mononuclear cells (PBMC) cells from the patient;
isolating CD14 positive monocytes from the PBMC sample;
maturing the CD14 positive monocytes into mature dendritic cells;
co-cultivating the mature dendric cells with isolated T cells to activate CD8+ T cells and cytotoxic T lymphocytes activity;
providing a sample of a photo-inactivated Leishmania;
mixing the mature dendritic cells with the Leishmania sample to form a vaccination solution; and,
delivering an effective amount of the vaccination solution to the patient.

16. The method of treating a solid tumor of claim 15 wherein the step of maturing the CD14 positive monocytes comprises exposure to GM-CSF and IL-4.

17. The method of treating a solid tumor of claim 16 wherein the step of maturing the CD14 positive monocytes comprises exposure to a lipopolysaccharide.

18. The method of treating a solid tumor of claim 15 wherein the photo-inactivated Leishmania are transgenically modified to render them susceptible to porphyrinogenesis.

19. The method of treating a solid tumor of claim 15 wherein the photo-inactivated Leishmania are transgenically modified to express alpha-enolase.

20. The method of treating a solid tumor of claim 15 wherein the photo-inactivated Leishmania are transgenically modified with a dual suicidal mechanism.
